(19) United States
(12) Patent Application Publication
(10) Pub. No.: US 2014/0128752 A1
(43) Pub. Date: May 8, 2014

(54) AMPLIFYING ORIENTATION CHANGES FOR ENHANCED MOTION DETECTION BY A MOTION SENSOR

(71) Applicant: Thomas Alan Donaldson, London (GB)

(72) Inventor: Thomas Alan Donaldson, London (GB)

(73) Assignee: AiphCom, San Francisco, CA (US)

(21) Appl. No.: 13/827,754

(22) Filed: Mar. 14, 2013

Related U.S. Application Data
(60) Provisional application No. 61/724,197, filed on Nov. 8, 2012.

(51) Int. Cl. A61B 5/024; A61B 5/0215; A61B 5/11

(52) U.S. Cl. CPC A61B 5/02438; A61B 5/11; A61B 5/0215
USPC 600/490; 600/595; 600/500; 600/508

(57) ABSTRACT

Techniques associated with amplifying orientation changes for enhanced motion detection by a motion sensor are described, including structures configured to enhance detection of motion, the structure having an articulator configured to amplify a motion and a pin configured to apply a force on a pivot point on the articulator, a motion sensor coupled to the structure and configured to detect motion of the structure, and circuitry configured to translate data associated with rotational motion of the articulator into a movement of an adjacent surface. In some embodiments, a method includes coupling a motion sensor to a skin surface using an articulator, the articulator configured to rotate in multiple planes, detecting rotational motion of the articulator using the motion sensor, and deriving data associated with movement on the skin surface.

### CROSS-REFERENCE TO RELATED APPLICATIONS

This application claims the benefit of U.S. Provisional Patent Application No. 61/724,197 (Attorney Docket No. ALI-157P), filed Nov. 8, 2012, which is incorporated by reference herein in its entirety for all purposes.

### FIELD

The present invention relates generally to electrical and electronic hardware, electromechanical and computing devices. More specifically, techniques related to amplifying orientation changes for enhanced motion detection by a motion sensor are described.

### BACKGROUND

Conventional devices and techniques for motion detection are limited in a number of ways. Conventional implementations of motion sensors, such as accelerometers, are not well-suited for accurately detecting and measuring movement having a small linear acceleration, as may occur by displacement of a skin surface in response to a pulse in a blood vessel. In particular, accelerometers typically have a threshold sensitivity and have a difficult time measuring translations that result in accelerations close to that threshold sensitivity.

Thus, what is needed is a solution for amplifying orientation changes for enhanced motion detection by a motion sensor without the limitations of conventional techniques.

### BRIEF DESCRIPTION OF THE DRAWINGS

Various embodiments or examples (“examples”) are disclosed in the following detailed description and the accompanying drawings:

FIG. 1 illustrates an exemplary structure for enhancing motion detection;

FIG. 2 illustrates an alternative exemplary structure for enhancing motion detection;

FIG. 3 illustrates another alternative exemplary structure for enhancing motion detection;

FIG. 4 is a diagram depicting the use of wearable devices equipped with enhanced motion detection;

FIG. 5 is a diagram illustrating an exemplary motion sensor changing orientation;

FIG. 6 is a diagram illustrating exemplary planes of orientation;

FIGS. 7A-7B illustrate exemplary articulators;

FIGS. 8A-8C illustrate exemplary articulator shapes;

FIG. 9 illustrates an exemplary configuration for coupling a motion sensor, circuitry, and a structure for enhancing motion detection;

FIG. 10 illustrates an exemplary funnel structure for enhancing motion detection;

FIG. 11 is a diagram depicting placement of an exemplary structure for enhancing motion detection adjacent to a skin surface;

FIG. 12 is another diagram depicting placement of an exemplary structure for enhancing motion detection adjacent to a skin surface;

FIG. 13 illustrates an exemplary structure for amplifying orientation changes for enhancing motion detection;

FIG. 14 illustrates an alternative exemplary structure for amplifying orientation changes for enhancing motion detection;

FIG. 15 illustrates another alternative exemplary structure for amplifying orientation changes for enhancing motion detection;

FIG. 16 illustrates different exemplary structure for amplifying orientation changes for enhancing motion detection;

FIG. 17 illustrates another different exemplary structure for amplifying orientation changes for enhancing motion detection;

FIG. 18 is a diagram showing another exemplary structure for amplifying orientation changes for enhancing motion detection;

FIGS. 19A-19B are diagrams depicting placement of exemplary articulators for amplifying orientation changes for enhancing motion detection;

FIGS. 20A-20C illustrate an exemplary structure for directing movement of a motion sensor; and

FIG. 21 is a graph illustrating an exemplary measured acceleration over time of movement caused by a pulse.

### DETAILED DESCRIPTION

Various embodiments or examples may be implemented in numerous ways, including as a system, a process, an apparatus, a device, and a method for enhanced motion detection. In some embodiments, motion may be detected using an accelerometer that responds to an applied force and produces an output signal representative of the acceleration (and hence in some cases a velocity or displacement) produced by the force. Embodiments may be used to detect the motion of a sub-component of a system. Techniques described are directed to systems, apparatuses, devices, and methods for using accelerometers, or other devices capable of detecting motion, to detect the motion of an element or part of an overall system. In some examples, the described techniques may be used to accurately and reliably detect the motion of a part of the human body or an element of another complex system. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

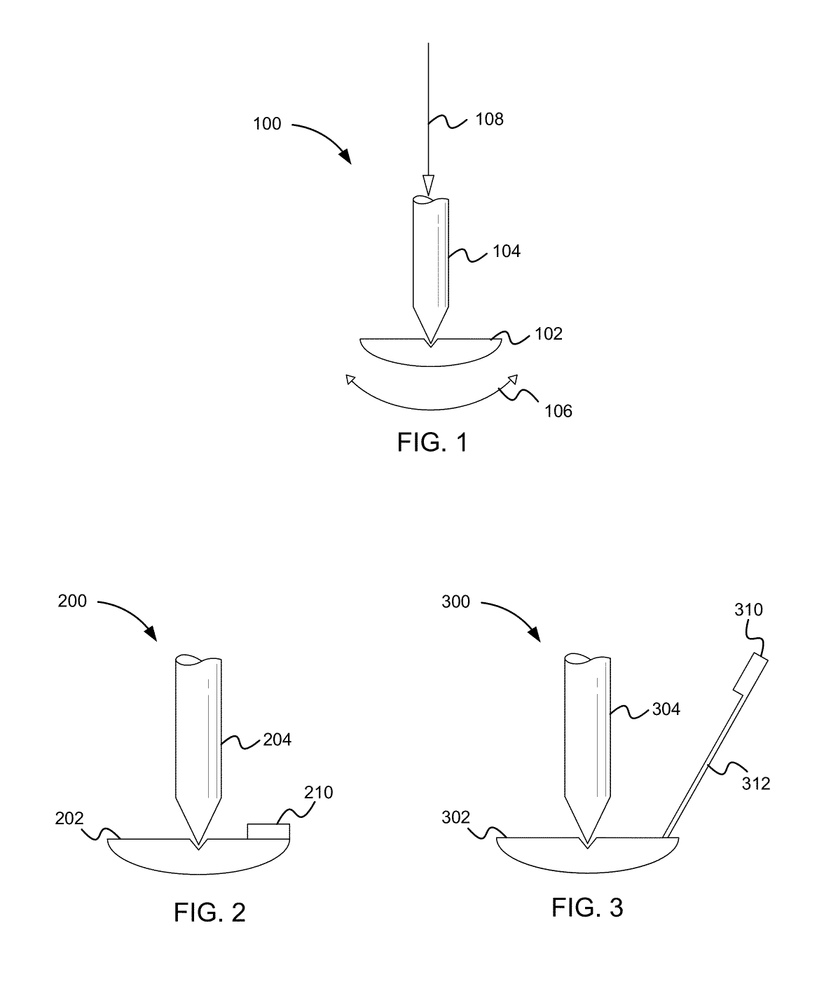

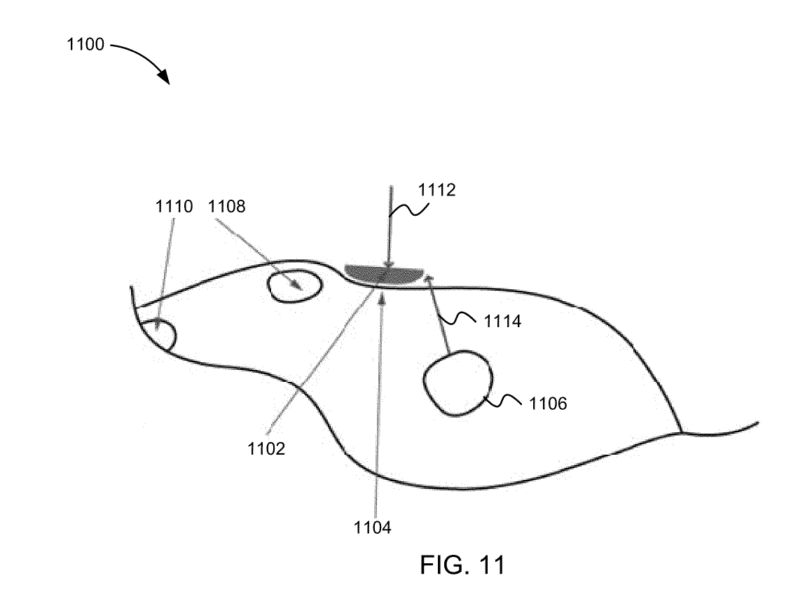

FIG. 1 illustrates an exemplary structure for enhancing motion detection. Here, structure 100 includes articulator (i.e., applicator) 102 and pin 104. As used herein, the terms “articulator” and “applicator” can be used, at least in some embodiments, interchangeably to refer to a structure suitable for applying, or placing, onto a surface (e.g., skin or other surface), to which a motion sensor may be coupled. In some examples, articulator 102 may be configured to transfer energy, for example rotational energy, from skin or another surface to a motion sensor. Here, articulator 102 may be formed using metal, plastic, or other suitable materials (i.e., holds a shape and compatible with skin). In some examples, articulator 102 may be configured to amplify rotational motion (i.e., orientation changes) or to amplify linear motion by converting or translating the linear motion into rotational motion. In some examples, pin 104 may apply force 108 to articulator 102. As shown, pin 104 may have a pointed end that fits into a correspondingly-shaped indentation in articulator 102, for example on a pivot point (i.e., at the center of a side or on an axis of rotation) of articulator 102, so that pin 104 may apply force 108 to articulator 102 without applying moment, torque, or any rotational force, to articulator 102. In some examples, structure 100 may rotate along rotation 106. For example, force 108 may be applied to one side of articulator 102 in order to hold another side of articulator 102 against skin, while allowing the another side of articulator 102 to register movement along adjacent skin by rotating along rotation 106. In other examples, articulator 102 may rotate differently than along rotation 106. For example, articulator 102 may be configured to rotate two or more planes. In some examples, articulator 102 may be configured to translate small amount of linear movement (i.e., near a threshold sensitivity of an accelerometer) in a blood vessel into a rotational movement more easily detected by a motion sensor (e.g., motion sensors 210 and 310 in FIGS. 2 and 3, respectively) coupled to articulator 102. For example, articulator 102 may be placed (and held) against a surface of skin adjacent to tissue, which in turn is adjacent to a blood vessel (see, e.g., FIGS. 11-12 and 19A-20). A pulse (i.e., pulse wave) of blood through such a blood vessel may have a small amount of linear movement that may be transferred through tissue to a skin surface against which articulator 102 may be placed such that articulator 102 may rotate in response to the movement of the blood vessel (see, e.g., FIGS. 11-12 and 19A-20). In other examples, the quantity, type, function, structure, and configuration of the elements shown may be varied and are not limited to the examples provided.

FIG. 2 illustrates an alternative exemplary structure for enhancing motion detection. Here, structure 200 includes articulator 202, pin 204 and motion sensor 210.

Like-numbered and named elements may describe the same or substantially similar elements as those shown in other descriptions. In some examples, pin 204 may be configured with a tip (i.e., pointed tip) that fits into a correspondingly-shaped indentation in articulator 202, for example on a pivot point (i.e., at the center of a side or on an axis of rotation) of articulator 102, so that pin 204 may be placed onto articulator 202 to apply a force to articulator 202 holding articulator 202 against a surface (e.g., skin or other surface) without applying moment. For example, articulator 202 may freely rotate in a multiple planes in response to movement on the surface against which it is being held.

In some examples, motion sensor 210 may be, or include, an accelerometer, a vibration sensor (e.g., acoustic, piezoelectric, or the like), a gyroscopic sensor, or other type of motion sensor. In some examples, motion sensor 210 may be coupled to articulator 202 by being mounted, or otherwise placed securely, onto articulator 202. In some examples, motion sensor 210 may be coupled to articulator 202 at or near an edge farther or farthest out from pin 204 so that motion sensor 210 may be subjected to, and thereby register, a greater amount of rotation, or other movement. In some examples, motion sensor 210 may be configured to register, or sense, rotational energy from articulator 202. For example, movement on a surface against which articulator 202 is being held may cause articulator 202 to rotate in one or more planes. In this example, motion sensor 210 may register and measure various characteristics (e.g., acceleration, direction, or the like) of the rotation of articulator 202. In some examples, articulator 202 may be configured to translate small amount of linear movement (i.e., near a threshold sensitivity of an accelerometer) in a blood vessel into a rotational movement more easily detected by motion sensor 210. For example, articulator 202 may be placed (and held) against a surface of skin adjacent to tissue, which in turn is adjacent to a blood vessel (see, e.g., FIGS. 11-12 and 19A-20). A pulse of blood through such a blood vessel may have a small amount of linear movement that may be transferred through tissue to a skin surface against which articulator 202 may be placed such that articulator 202 may rotate in response to the movement of the blood vessel (see, e.g., FIGS. 11-12 and 19A-20), and motion sensor 210 may capture the rotation of articulator 202. In other examples, the quantity, type, function, structure, and configuration of the elements shown may be varied and are not limited to the examples provided.

FIG. 3 illustrates another alternative exemplary structure for enhancing motion detection. Here, structure 300 includes articulator 302, pin 304, motion sensor 310 and post 312. Like-numbered and named elements may describe the same or substantially similar elements as those shown in other descriptions. In some examples, post 312 may be mounted, or otherwise placed securely, onto articulator 302. In some examples, post 312 may be configured to couple motion sensor 310 to articulator 302. In some examples, post 312 may be configured to extend outward from an edge of articulator 302, and away from a pivot point (i.e., an axis of rotation) of articulator 302, such that motion sensor 310 may be subjected to, and thereby register, a greater amount of rotation when articulator 302 rotates in response to movement on a surface against which articulator 302 is being held. In some examples, motion sensor 310 may be configured to register, or sense, rotational energy from articulator 302. For example, movement on a surface against which articulator 302 is being held may cause articulator 302 to rotate in one or more planes. In this example, motion sensor 310 may register and measure various characteristics (e.g., acceleration, direction, or the like) of the rotation of articulator 302. In some examples, articulator 302 may be configured to translate small amount of linear movement (i.e., near a threshold sensitivity of an accelerometer) in a blood vessel into a rotational movement more easily detected by motion sensor 310. For example, articulator 302 may be placed (and held) against a surface of skin adjacent to tissue, which in turn is adjacent to a blood vessel (see, e.g., FIGS. 11-12 and 19A-20). A pulse of blood through such a blood vessel may have a small amount of linear movement that may be transferred through tissue to a skin surface against which articulator 302 may be placed such that articulator 302 may rotate in response to the movement of the blood vessel (see, e.g., FIGS. 11-12 and 19A-20), and motion sensor 310 may capture the rotation of articulator 302. In other examples, the quantity, type, function, structure, and configuration of the elements shown may be varied and are not limited to the examples provided.

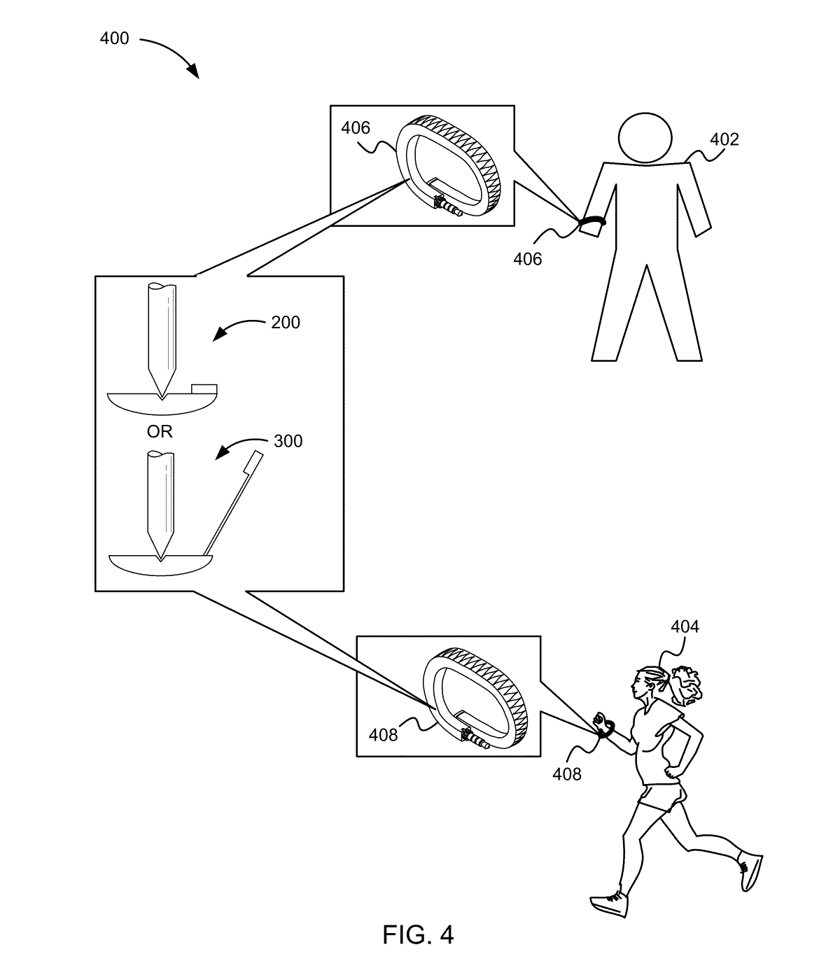

FIG. 4 is a diagram depicting the use of wearable devices equipped with enhanced motion detection. Here, diagram 400 includes users 402-404, wearable devices 406-408, and structures 200-300. Like-numbered and named elements may describe the same or substantially similar elements as those shown in other descriptions. As shown, wearable device 406 may be worn by user 402, and wearable device 408 may be worn by user 404. In some examples, wearable devices 406-408 may be implemented as a band having one or more sensors, including motion sensors. In some examples, wearable devices 406-408 may include motion sensors configured to register and process data associated with greater movement, for example the movement of user 404, as well as smaller movement, for example the movement of user 402. In some examples, wearable device 406-408 may be implemented with structure 200 or structure 300 to enhance detection of motion by a motion sensor, as described herein. In some examples, wearable devices 406-408 may be implemented with circuitry, logic, software and/or processing capabilities to distinguish between different types of motion data, for example, to identify data associated with motion caused by a user's gait or physical activity from data associated with motion caused by a user's heartbeat or pulse. In some examples, wearable devices 406-408 also may be configured to process data from a motion sensor coupled to structures 200-300 to derive data associated with movement on an adjacent skin surface (e.g., on users 402-404's wrists, arms, or other body parts). For example, wearable devices 406-408 may be configured to derive data associated with a direction of movement on an adjacent skin surface, a magnitude of a force exerted by a pulse in a blood vessel underneath an adjacent skin surface, a time period between two pulses, a heart rate, a blood pressure, or the like. In other examples, the quantity, type, function, structure, and configuration of the elements shown may be varied and are not limited to the examples provided.

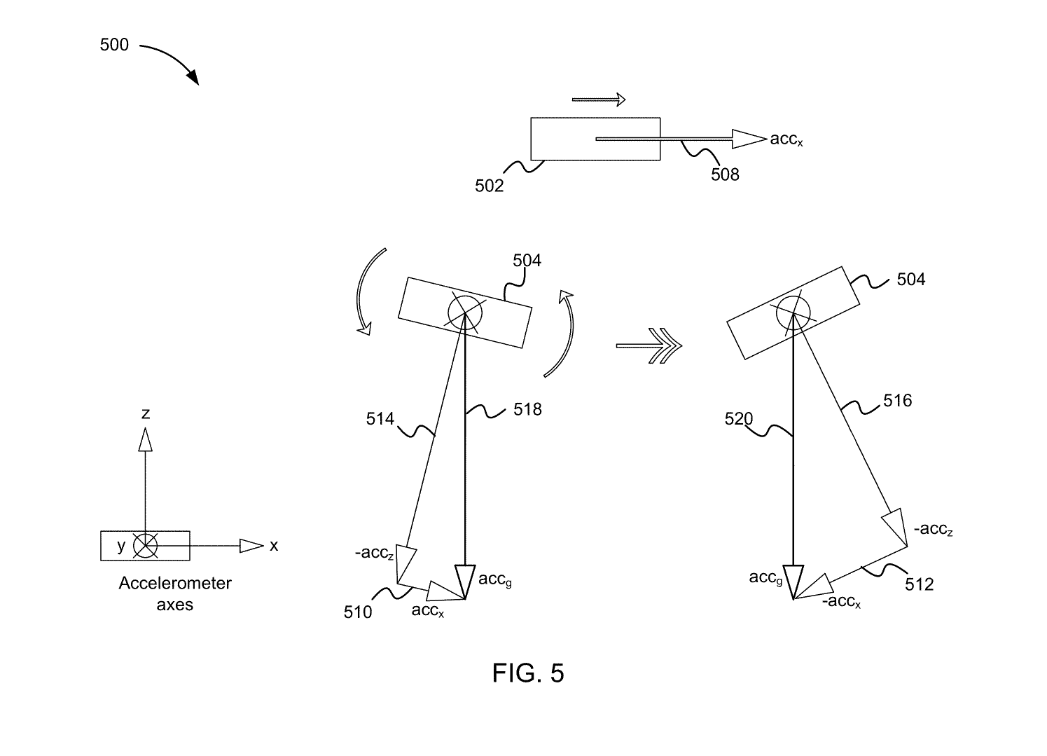

FIG. 5 is a diagram illustrating an exemplary motion sensor changing orientation. Here, diagram 500 includes motion sensors 502-504, x-axis acceleration 508-512, z-axis acceleration 514-516, and gravitational acceleration 518-520. Like-numbered and named elements may describe the same or substantially similar elements as those shown in other descriptions. In some examples, x-axis acceleration 508, to which motion sensor 502 may be subject to, may be a linear or translational acceleration. In some examples, the linear or translational movement giving rise to x-axis acceleration 508 may be converted into rotation, for example by mounting motion sensors 502-504 onto structures (e.g., as shown in at least FIGS. 1-3, 9, 11 and 13-18) configured to amplify motion. Then, as shown with motion sensor 504, changes in orientation of acceleration due to gravity (e.g., gravitational acceleration 518-520) relative to an orientation of motion sensor 504, as indicated by x-axis acceleration 510-512 and z-axis acceleration 514-516, gravity being large relative to the sensitivity of motion sensor 504. In other examples, the quantity, type, function, structure, and configuration of the elements shown may be varied and are not limited to the examples provided.

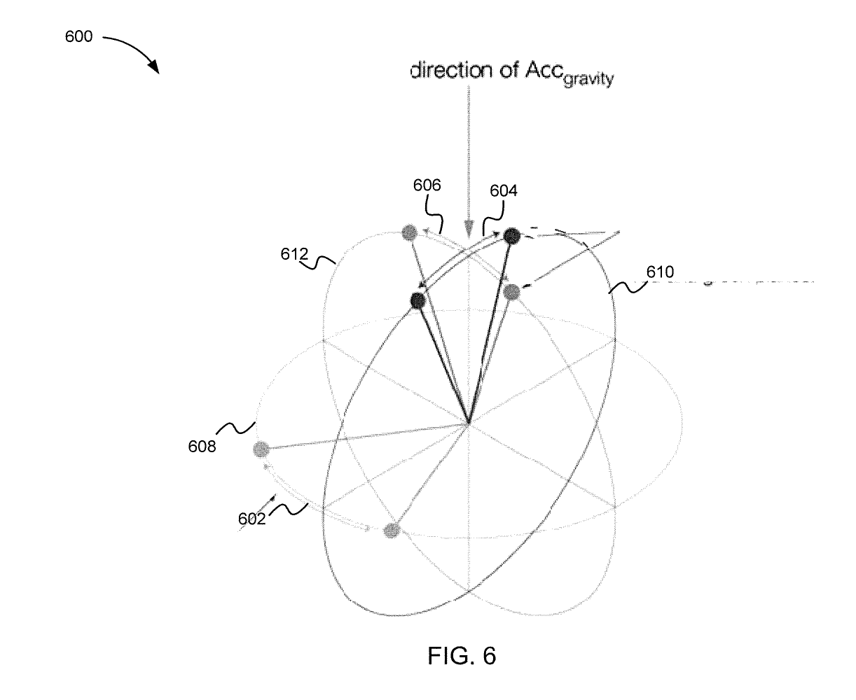

FIG. 6 is a diagram illustrating exemplary planes of orientation. Here, diagram 600 includes rotational directions 602-606 and planes 608-612. As shown, an object rotating in direction 602 is rotating in plane 608, an object rotating in direction 604 is rotating in plane 610, and an object rotating in direction 606 is rotating in plane 612. In this example, plane 608 is normal to gravity, and rotation in direction 602 may not provide gravitation advantage for detecting orientation changes, as described in FIG. 5. On the other hand, creating or causing rotation in planes 610-612 can provide the gravitation advantage for detecting orientation changes, as described in FIG. 5. In some examples, a motion sensor may be placed or mounted on an articulator (e.g., FIGS. 1-4, 7A-7B, 8A-8C, 11 and 13-18) configured to rotate in multiple planes, and thus to provide the gravitation advantage described in FIG. 5. In other examples, the quantity, type, function, structure, and configuration of the elements shown may be varied and are not limited to the examples provided.

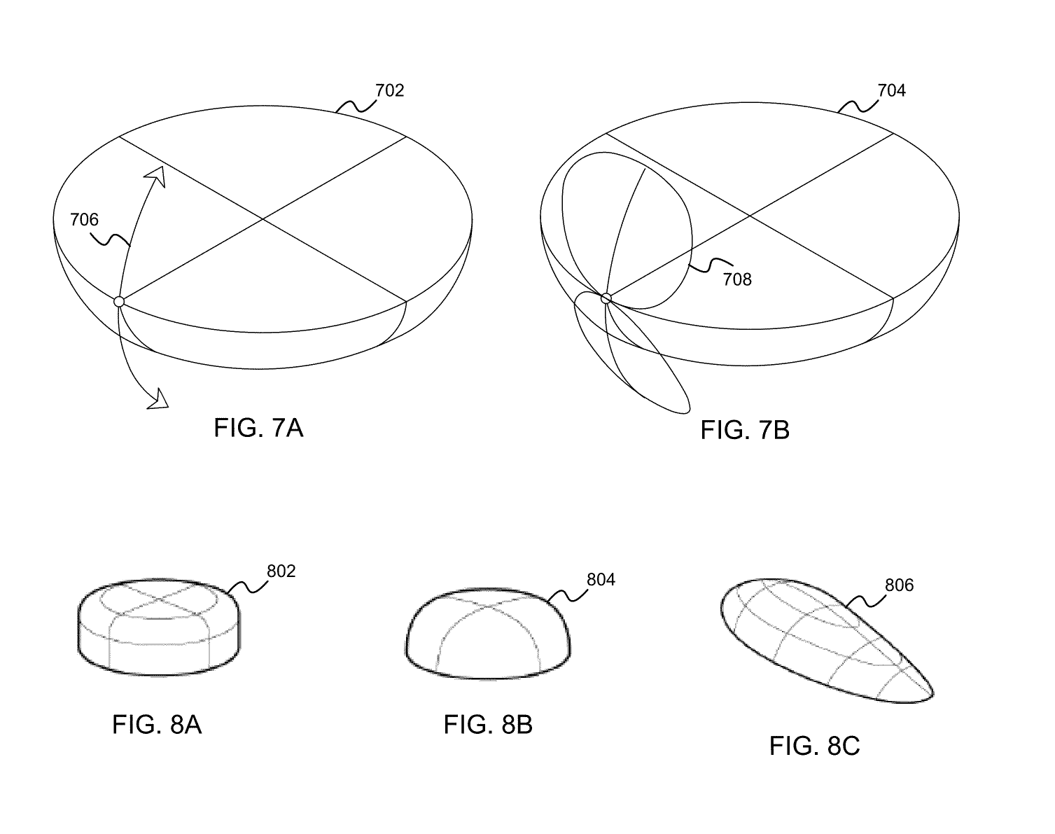

FIGS. 7A-7B illustrate exemplary articulators. In some examples, articulator 702 may be configured to move in directions 706 along a plane. In other examples, articulator 704 may be configured to move in directions 708 along two or more planes. As shown, articulators 702-704 may have a rounded surface for placing adjacent to, or contacting, a surface (i.e., a skin surface). In some examples, articulators 702-704 may be configured to rotate (e.g., in directions 706-708) in response to movement on a surface adjacent to the rounded surface of articulators 702-704. Instabilities in articulators 702-704 that cause orientation changes in two or more axes may assist in enhancing motion detection, for example, by exaggerating movement. Examples of articulator shapes that may give rise to such instabilities are shown in FIGS. 8A-8C, which show articulators 802-806. In some examples, articulators 802-806 may be configured to be placed against a surface (e.g., skin surface or the like) such that movement on said surface causes articulators 802-806 to roll, or otherwise cause a rotational force. In some examples, articulators 802-806 may be shaped to minimize deformation of a surface against which articulators 802-806 may be held. In particular, articulators 802-806 may be shaped to reduce edges or corners (which may stretch or stress skin thereby changing skin tension) on a side that contacts a skin surface, such that the skin's movement associated with a pulse is not dampened, or otherwise reduced or changed. For example, articulator 802 has filleted or rounded edges on one side. In another example, articulator 804 has no edges on one side, the one side being substantially round, or semispherical. In still another example, articulator 806 has an asymmetrical, rounded shape configured to cause orientation changes in a plurality of planes. In other examples, the quantity, type, function, structure, and configuration of the elements shown may be varied and are not limited to the examples provided.

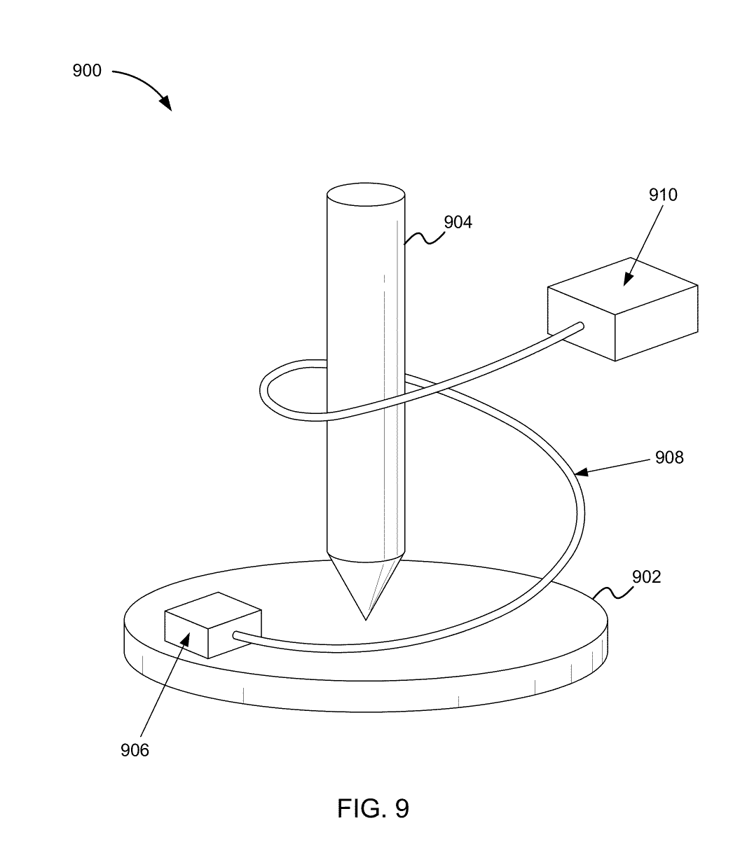

FIG. 9 illustrates an exemplary system for coupling a motion sensor, circuitry, and a structure for enhancing motion detection. Here, system 900 includes articulator 902, pin 904, sensor 906, wire 908 and circuitry 910. Like-numbered and named elements may describe the same or substantially similar elements as those shown in other descriptions. In some examples, articulator 902 may be shaped similar to the shapes shown in FIGS. 1-4, 7A-7B and 8A-8C. In other examples, articulator 902 may be shaped differently. In some examples, sensor 906 may be a motion sensor (e.g., motion sensors 210, 310, 1014, 1112, 1610 and 1710 in FIGS. 2, 3, 10, 11, 16 and 17, respectively), and may be placed (i.e., mounted) on or near an edge of articulator 902 far from a pivot point of articulator 902 (see, e.g., FIG. 2). In other examples, sensor 906 may be coupled to articulator 902 differently (see, e.g., FIG. 3). In some examples, sensor 906 may be coupled to circuitry 910 using wire 908. In some examples, wire 908 may be configured to enable the transfer or communication of data between sensor 906 and circuitry 910, for example by allowing an electrical, or other type of, signal to pass through. In some examples, wire 908 may have a coil form, or may be able to be manipulated into a coil. In some examples, wire 908 may comprise a stress-relieving coil of wire. In other examples, sensor 906 and circuitry 910 may be coupled differently, for example, wirelessly. In some examples, circuitry 910 may be mounted to a wearable device (e.g., wearable devices 406-408 in FIG. 4). In some examples, circuitry 910 may be configured to process data received from sensor 906. For example, circuitry 910 may be configured to translate data associated with rotational motion of articulator 902, as detected by sensor 906, into data associated with linear motion of an adjacent structure (e.g., a blood vessel or other tissue). In another example, circuitry 910 may be configured to derive additional data using sensor data from sensor 906, as well as other data from databases, other sensors, and/or other devices. In other examples, the quantity, type, function, structure, and configuration of the elements shown may be varied and are not limited to the examples provided.

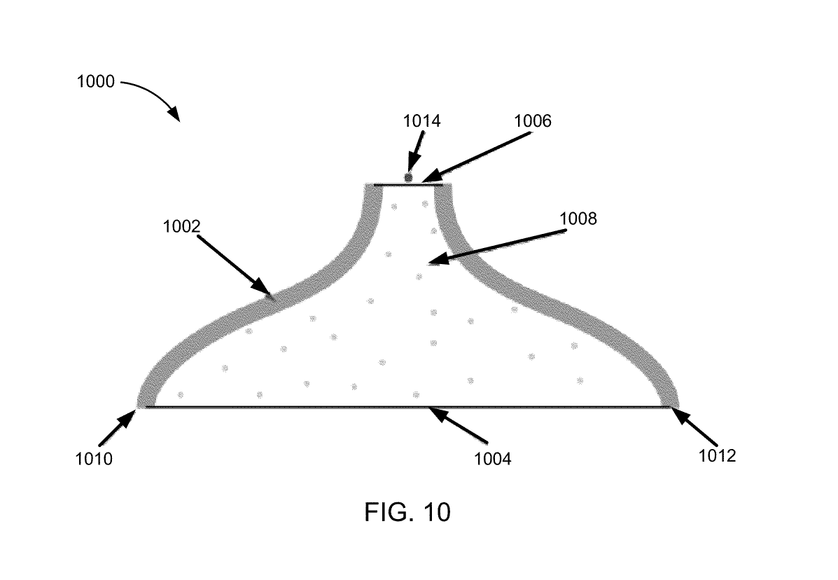

FIG. 10 illustrates an exemplary funnel structure for enhancing motion detection. Here, structure 1000 includes funnel 1002, large diaphragm 1004, small diaphragm 1006, fluid 1008, edges 1010-1012, and motion sensor 1014. Like-numbered and named elements may describe the same or substantially similar elements as those shown in other descriptions. In some examples, structure 1000 may be configured to transmit a force from a larger area to a smaller area. In some examples, large diaphragm 1004 may be placed against or adjacent to a surface (i.e., skin surface), and may be configured to move in response to movement on said surface. For example, diaphragm 1004 may be formed using a deformable material (e.g., rubber, plastic, other materials having material memory, or the like). On the other hand, funnel 1002 may be formed using a stiffer material, and thus edges 1010-1012 may be stiffer relative to diaphragms 1004-1006. In some examples, funnel 1002 may be configured to hold or contain a liquid (viscous or otherwise), such as fluid 1008. Deformations in large diaphragm 1004 may travel through fluid 1008, being funneled by funnel 1002, and echo in small diaphragm 1006, the displacement of which may then be sensed using motion sensor 1014. In some examples, diaphragm may be placed directly onto a skin surface, and edges 1010-1012 may be held against such skin surface to occlude (i.e., hold, trap, keep or place) a blood vessel (i.e., through skin tissue), for example, against a bone, tendon, or other tissue structure. In other examples, the quantity, type, function, structure, and configuration of the elements shown may be varied and are not limited to the examples provided.

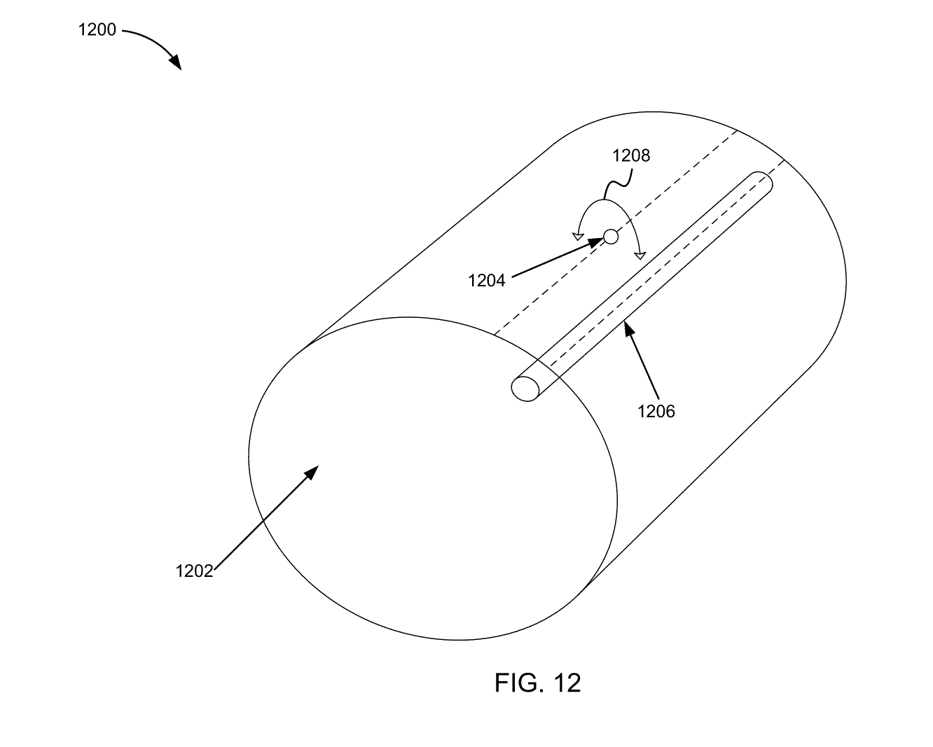

FIG. 11 is a diagram depicting placement of an exemplary structure for enhancing motion detection adjacent to a skin surface. Here, diagram 1100 includes articulator 1102, skin surface 1104, blood vessel 1106, tendons 1108-1110, and forces 1112-1114. Like-numbered and named elements may describe the same or substantially similar elements as those shown in other descriptions. In some examples, blood vessel 1106 may be an artery through which a pulse may travel. In other examples, blood vessel 1106 may be a vein, capillary, or other part of the circulatory system. In some examples, articulator 1102 may be held against skin surface 1104 by a force 1112, for example using a pin-like structure (e.g., pins 104, 204, 304 and 904 in FIGS. 1-3 and 9, respectively), creating a dip in skin surface 1104 between tendon 1108 and blood vessel 1106. In some examples, force 1112 may be directed onto a pivot point, or on an axis of rotation, on a side of articulator 1102 opposite to the skin adjacent side. In some examples, force 1112 may be of sufficient magnitude to form a dip in skin surface 1104 that pushes fat tissue or other subcutaneous tissue away to improve the response of articulator 1102 to force 1114. In some examples, force 1112 may be configured (i.e., located and provided with sufficient magnitude) to occlude blood vessel 1106 against a bone tissue (e.g., a radius in a wrist). As shown in FIG. 12, the placement of articulator 1102 between tendon 1108 and blood vessel 1106 may increase the rotation of articulator 1102 in response to force 1114 by allowing force 1114 to act on articulator 1102 with a tangential or circumferential force. In some examples, force 1114 may be caused by a pulse running through blood vessel 1106. In some examples, force 1114 may act as a radial force, causing a moment about a pivot point, or on axis of rotation, of articulator 1102, thereby causing articulator 1102 to rock, rotate, or otherwise move about the pivot. In some examples, articulator 1102 may be implemented with a motion sensor (e.g., motion sensors 210, 310, 1014, 1112, 1610 and 1710 in FIGS. 2, 3, 10, 11, 16 and 17, respectively) to register (i.e., sense) the rotational acceleration resulting from the movement of articulator 1102 in response to force 1114. In other examples, other motion sensors may be implemented on or near the skin surface and articulator 1102 to detect orientation change (or other motion) not caused by a pulse. For example, a second motion sensor (not shown) may be placed elsewhere on the same skin surface or body part (i.e., on the other side of tendon 1110) to detect and measure orientation change (or other motion) of the skin surface or body part unrelated to motion caused by blood vessel 1106. In this example, data from the second motion sensor may be used to cancel, or subtract, out a portion of sensor data detected using articulator 1102 that may not be attributable to a pulse in blood vessel 1106, and thereby determine the attributes associated with said pulse. In other examples, a first motion sensor may be implemented to detect and measure the motion of articulator 1102 only when a second motion sensor determines that a body part, which articulator 1102 is in contact with or adjacent to, is in a good state for such measurements. For example, if a first motion sensor and articulator 1102 are configured for detection and measurement of pulse-related information, a second motion sensor may determine when a wrist, to which the first motion sensor and articulator 1102 is coupled, is at rest. When the wrist is not at rest, the data from the first motion sensor may not be considered or used in (i.e., to derive information such as heart rate). In still other examples, the quantity, type, function, structure, and configuration of the elements shown may be varied and are not limited to the examples provided.

FIG. 12 is another diagram depicting placement of an exemplary structure for enhancing motion detection adjacent to a skin surface. Here, diagram 1200 includes limb (i.e., cross-section) 1202, articulator 1204, blood vessel 1206 and rotation direction 1208. Like-numbered and named elements may describe the same or substantially similar elements as those shown in other descriptions. In some examples, limb 1202 may be a wrist and blood vessel 1206 may be an artery below the skin surface of the wrist. In some examples, articulator 1204 may be placed in a location offset from blood vessel 1206, for example along an axis parallel to blood vessel 1206, such that movement from a pulse through blood vessel 1206 may act tangentially or circumferentially on articulator 1204 (e.g., to cause rotation in at least a plane perpendicular to blood vessel 1206). In other examples, the quantity, type, function, structure, and configuration of the elements shown may be varied and are not limited to the examples provided.

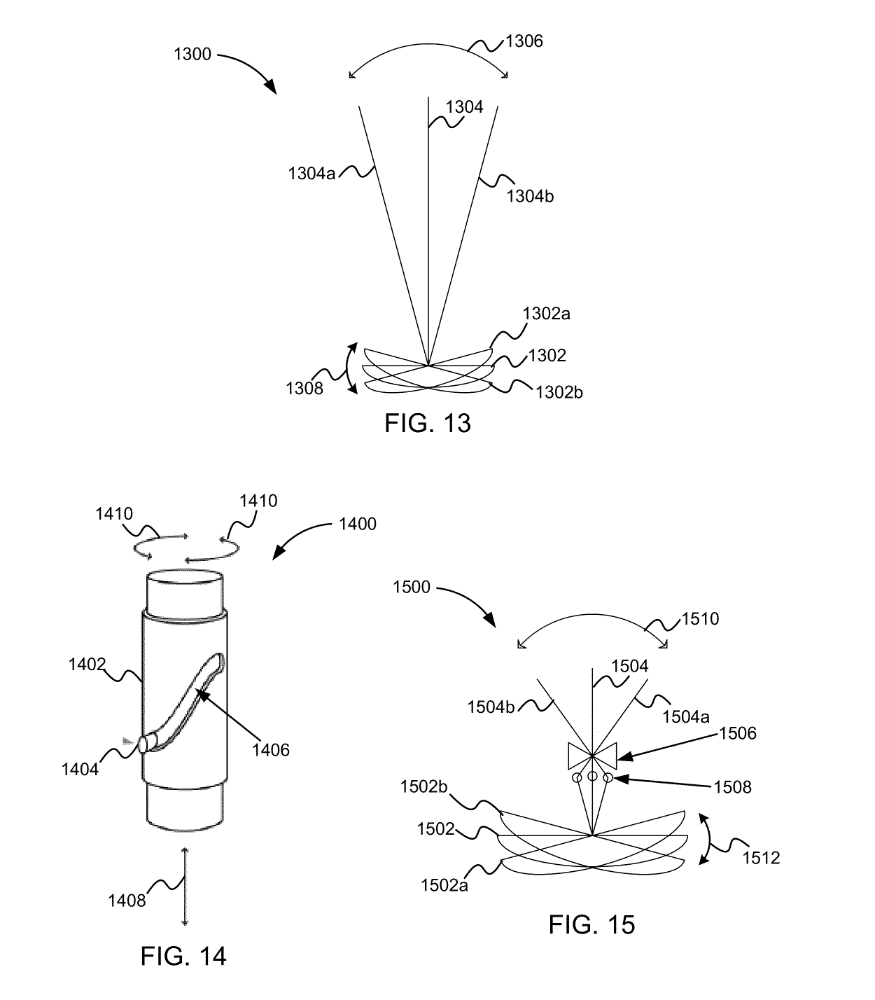

FIG. 13 illustrates an exemplary structure for amplifying orientation changes for enhancing motion detection. Here, structure 1300 includes articulator 1302, lever 1304 and rotations 1306-1308. Like-numbered and named elements may describe the same or substantially similar elements as those shown in other descriptions. In some examples, lever 1304 may be a rigid bar with one end placed on a pivot point, or on an axis of rotation, of articulator 1302. In some examples, when articulator 1302 moves to position 1302a, lever 1304 will move correspondingly to position 1304a, and when articulator 1304 moves to position 1302b, lever 1304 will move correspondingly to position 1304b. Thus, when articulator moves according to rotation 1308 (i.e., the acceleration and distance of rotation 1308), an end of lever 1304 not attached to articulator 1302 (i.e., a free end of lever 1304) moves according to rotation 1306 (i.e., the acceleration and distance of rotation 1306). In some examples, lever 1304 may be longer than a diameter of articulator 1302, and thus rotation 1308 has a greater rotational acceleration than rotation 1306. In some examples, a motion sensor (e.g., motion sensors 210, 310, 1014, 1112, 1610 and 1710 in FIGS. 2, 3, 10, 11, 16 and 17, respectively) may be coupled to a free end of lever 1304 to detect motion at the free end. Thus, where articulator 1302 is placed on or adjacent to a surface wherein a movement in the surface is sufficient to cause articulator 1302 to rotate as indicated by rotation 1308, a motion sensor implemented at a free end of lever 1304 may register (i.e., detect) and measure rotation 1306, thereby amplifying the movement (i.e., using orientation changes). In other examples, the quantity, type, function, structure, and configuration of the elements shown may be varied and are not limited to the examples provided.

FIG. 14 illustrates an alternative exemplary structure for amplifying orientation changes for enhancing motion detection. Here, structure 1400 includes housing 1402, pin 1404, slot 1406, direction 1408 and rotation 1410. Like-numbered and named elements may describe the same or substantially similar elements as those shown in other descriptions. In some examples, slot 1406 may comprise a narrow opening or indentation on the side of housing 1402, which has a cylindrical shape. In some examples, pin 1404 may be a stationary pin constrained within slot 1406, such that when housing 1402 moves in direction 1408, stationary pin 1404 slides along the slot causing housing 1402 to rotate about an axis as indicated by rotation 1410. Thus, structure 1400 may convert a linear movement (i.e., no orientation change) into a rotation. In other examples, the quantity, type, function, structure, and configuration of the elements shown may be varied and are not limited to the examples provided.

FIG. 15 illustrates another alternative exemplary structure for amplifying orientation changes for enhancing motion detection. Here, structure 1500 includes articulator 1502, lever 1504, sliding joint 1506 and pivot 1508. Like-numbered and named elements may describe the same or substantially similar elements as those shown in other descriptions. In some examples, lever 1504 may comprise pivot 1508 at which lever 1504 may bend at an angle. In some examples, lever 1504 also may be pinned by sliding joint 1506, and may be configured to bend at a point where lever 1504 is pinned by sliding joint 1506. Where the distance along lever 1504 between sliding joint 1506 and pivot 1508 is small (i.e., smaller than the distance between sliding joint 1506 and a free end of lever 1504), movement of articulator 1502 may be amplified. For example, using the placement of articulator 1502, lever 1504, sliding joint 1508 and pivot 1508, as shown, movement of articulator 1502 from position 1502a to position 1502b may result in rotation 1512 at an edge of articulator 1502, and may result in rotation 1510 at a free end of articulator 1502. In other examples, the quantity, type, function, structure, and configuration of the elements shown may be varied and are not limited to the examples provided.

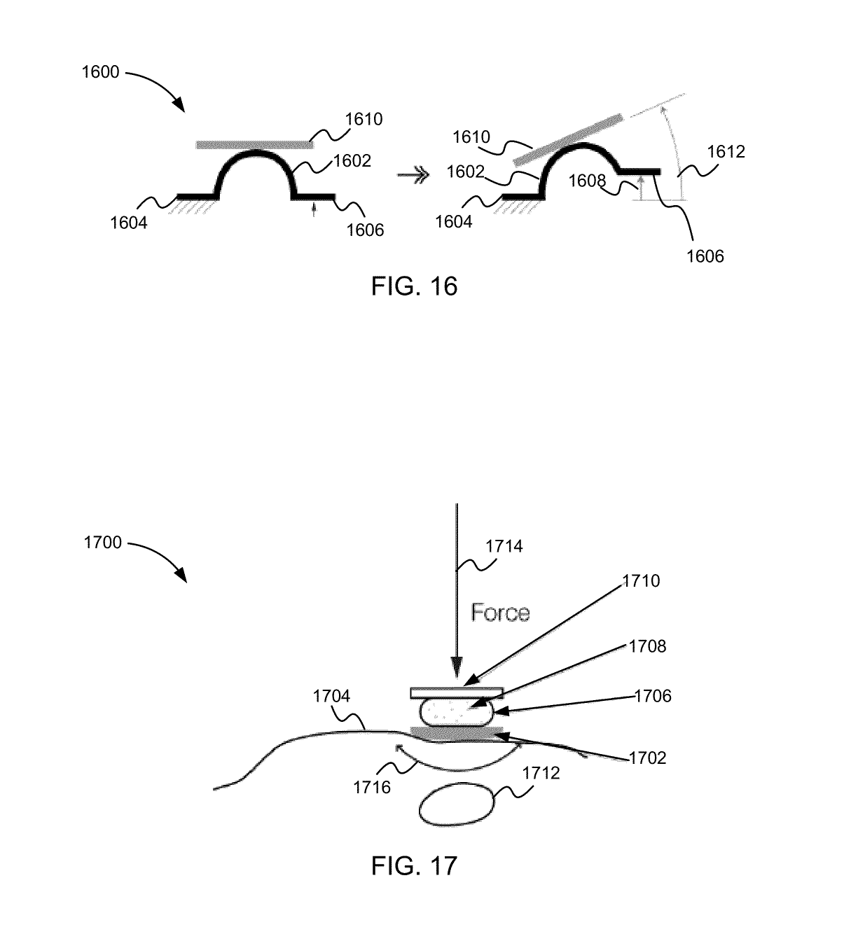

FIG. 16 illustrates different exemplary structure for amplifying orientation changes for enhancing motion detection. Here, structure 1600 includes hump 1602, footings 1604-1606, distance 1608, motion sensor 1610 and rotation 1612. Like-numbered and named elements may describe the same or substantially similar elements as those shown in other descriptions. In some examples, hump 1602 may be coupled to a surface using footings 1605-1606. In some examples, footing 1604 may be coupled to a housing, or other structure, while footing 1606 may be coupled to a skin surface, wherein footing 1606 may be displaced with movement on the skin surface, and footing 1604 may not. As shown, a displacement of footing 1606 of distance 1608 may result in a rotation 1612 of that may be registered (i.e., detected) and/or measured by motion sensor 1610. In other examples, the quantity, type, function, structure, and configuration of the elements shown may be varied and are not limited to the examples provided.

FIG. 17 illustrates another different exemplary structure for amplifying orientation changes for enhancing motion detection. Here, structure 1700 includes articulator 1702, skin surface 1704, bubble 1706, fluid 1708, motion sensor 1710, blood vessel 1712, force 1714 and rotation 1716. Like-numbered and named elements may describe the same or substantially similar elements as those shown in other descriptions. In some examples, articulator 1702 may be placed on or adjacent to skin surface 1704, and may be configured to move (e.g., rotate, rock, or the like) in response to movement by skin surface 1704, for example caused by a pulse traveling through blood vessel 1712. For example, a pulse through blood vessel 1712 may displace skin surface 1704, which may cause articulator 1702 to move according to rotation 1716. In some examples, articulator 1702 may be coupled to bubble 1706, which may be filled with fluid 1708. In some examples, fluid 1708 may be incompressible, such that rotational movement by articulator 1702 may be transferred through bubble 1706 to motion sensor 1710 without compression distortion by fluid 1708. In some examples, bubble 1706 may be formed of a flexible, but inelastic, material (e.g., plastic (i.e., thermoplastic elastomer), rubber, or the like). In other examples, the quantity, type, function, structure, and configuration of the elements shown may be varied and are not limited to the examples provided.

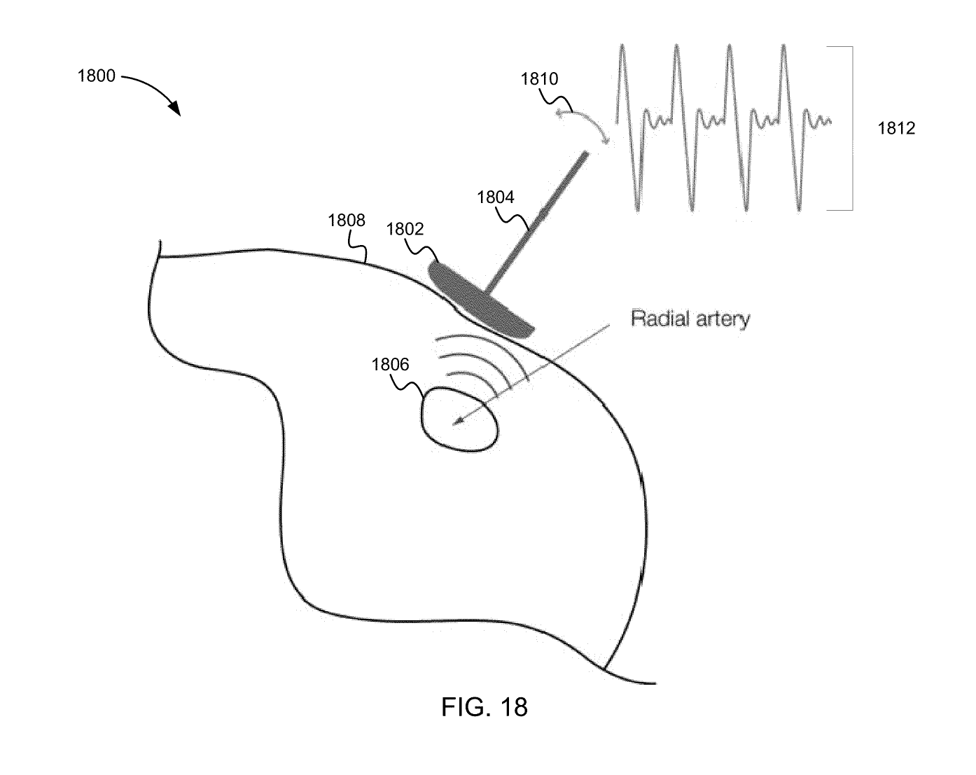

FIG. 18 is a diagram showing another exemplary structure for amplifying orientation changes for enhancing motion detection. Here, diagram 1800 includes articulator 1802, beam 1804, blood vessel 1806, skin surface 1808, direction 1810 and waveform 1812. Like-numbered and named elements may describe the same or substantially similar elements as those shown in other descriptions. In some examples, beam 1804 may be a resonant beam placed, mounted or otherwise coupled, to articulator 1802. In some examples, beam 1804 may be configured to oscillate (i.e., resonate) in response to a rotation in articulator 1802. For example, a pulse running through blood vessel 1806 may exert a force on articulator 1802 by moving skin surface 1808. In some examples, such a force may cause articulator 1802 to rotate in one or more planes. In an example, a rotation of articulator 1802 may cause beam 1804 to oscillate in direction 1810 at a frequency, represented by waveform 1812. In some examples, a motion sensor (e.g., motion sensors 210, 310, 1014, 1112, 1610 and 1710 in FIGS. 2, 3, 10, 11, 16 and 17, respectively) may be coupled to beam 1804 (i.e., mounted onto, or near a free end of, beam 1804) to detect a resonance in beam 1804 caused by a pulse in blood vessel 1806. In some examples, beam 1804 may resonate at a higher frequency, which may result in lower noise. In other examples, the quantity, type, function, structure, and configuration of the elements shown may be varied and are not limited to the examples provided.

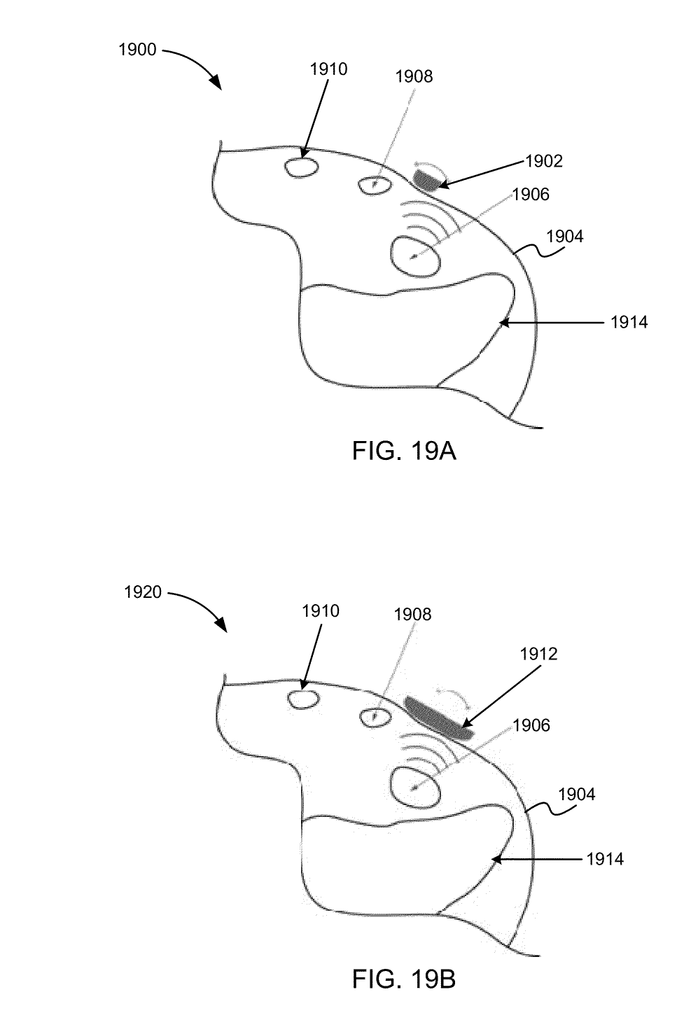

FIGS. 19A-19B are diagrams depicting placement of exemplary articulators for amplifying orientation changes for enhancing motion detection. Here, diagrams 1900 and 1920 include articulators 1902 and 1912, skin surface 1904, blood vessel 1906, tendons 1908-1910 and bone 1914. Like-numbered and named elements may describe the same or substantially similar elements as those shown in other descriptions. In some examples, blood vessel 1906 may be a radial artery, tendon 1908 may be a flexor carpi radialis, tendon 1910 may be a Palmaris longus, and bone 1914 may be a radius. A pulse traveling through blood vessel 1906 may act upon an articulator (e.g., articulators 1902 and 1912, or the like) placed on (i.e., against or adjacent to) skin surface 1904 at a location between tendon 1908 and blood vessel 1906. In some examples, articulators 1902 and 1912 may be configured (i.e., shaped) to rock or rotate in response to a pulse from blood vessel 1906, as described herein. In some examples, articulators 1902 and 1912 may be sized to fit in a dip in skin surface 1904 that may be formed between tendon 1908 and blood vessel 1906 when force is applied to press articulators 1902 and 1912 against skin surface 1904. In other examples, the quantity, type, function, structure, and configuration of the elements shown may be varied and are not limited to the examples provided.

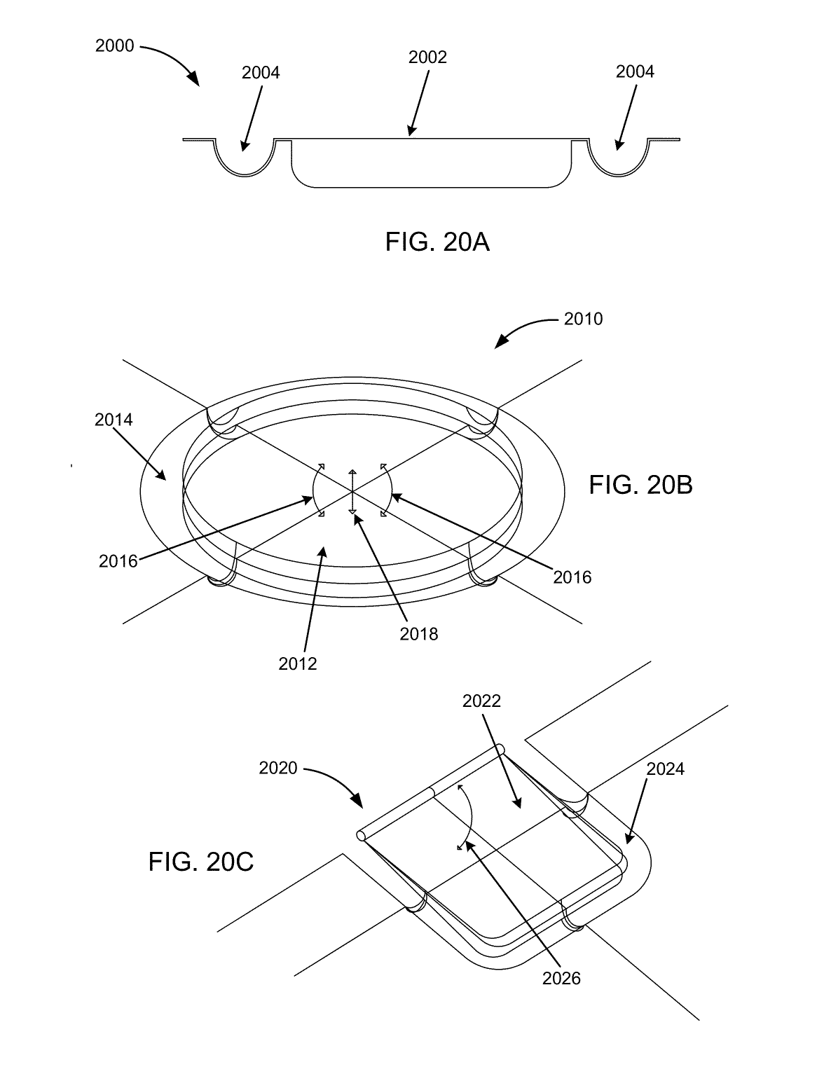

FIGS. 20A-20C illustrate an exemplary structure for housing a motion sensor. Here, structure 2000 includes motion sensor casing 2002 and canal 2004, structure 2010 includes motion sensor casing 2012 and canal 2014, and structure 2020 includes motion sensor casing 2022 and canal 2024. In some examples, canals 2004, 2014 and 2024 may be formed as part of structures 2000, 2010 and 2020, and may encircle partially or wholly motion sensor casings 2002, 2012 and 2022, respectively. In some examples, canals 2004, 2014 and 2024 may be filled with a material (e.g., treated cloth (i.e., fabric), rubber, plastic, foam, wood, or the like) that is rigid or has material memory (i.e., able to restore an original shape after being deformed), and be configured to provide a force that acts as a barrier to linear movement, instead directing motion sensors (not shown) to change orientation in response to other forces acting on structures 2000, 2010 and 2020. In some examples, a constraining force provided by canal 2014, and any material filling canal 2014, may direct a motion sensor to rotate in direction 2016 about axis 2018. In another example, a constraining force provided by canal 2024, and any material filling canal 2024, may direct a motion sensor to rotate in direction 2026. In other examples, the quantity, type, function, structure, and configuration of the elements shown may be varied and are not limited to the examples provided.

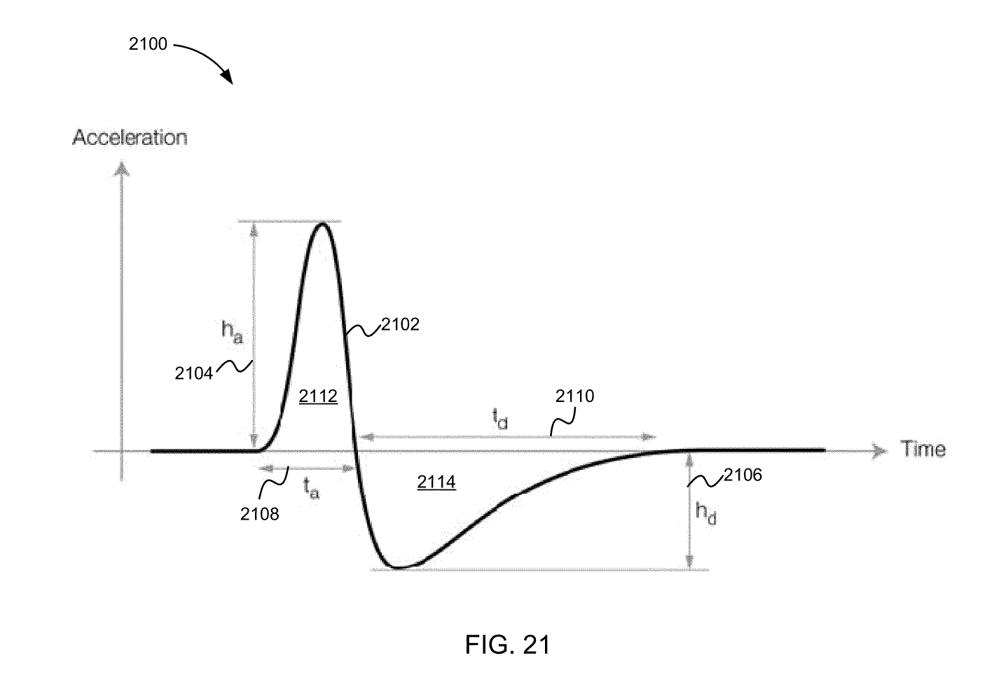

FIG. 21 is a graph illustrating an exemplary measured acceleration over time of movement caused by a pulse. Here, graph 2100 shows waveform 2102, heights 2104-2106, times 2108-2110 and volumes 2112-2114. Like-numbered and named elements may describe the same or substantially similar elements as those shown in other descriptions. In some examples, waveform 2102 may represent acceleration of movement of a blood vessel, or tissue adjacent to, or acted upon by, the blood vessel, over time as a result of a pulse (i.e., of blood pushed through the blood vessel by a heart beat). In some examples, height 2104 may represent a peak acceleration (i.e., in a positive direction) during an attack portion of waveform 2102. For example, the attack may last time 2108, and the attack portion of waveform 2102 may have a volume 2112. In some examples, height 2106 may represent a trough acceleration (i.e., acceleration in a negative or opposite direction) during a decay portion of waveform 2102. For example, the decay may last time 2110 and the decay portion of waveform 2102 may have volume 2114. Using the parameters provided by waveform 2102, information about blood pressure (i.e., pressure exerted by circulating blood on walls of a blood vessel) may be inferred. In other examples, the quantity, type, function, structure, and configuration of the elements shown may be varied and are not limited to the examples provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A device, comprising:
a structure configured to enhance detection of movement, the structure comprising an articulator configured to amplify a motion and a pin configured to apply a force on a pivot point on the articulator;
an accelerometer coupled to the structure and configured to detect motion of the structure; and
circuitry configured to translate data associated with rotational motion of the articulator to determine a movement of an adjacent surface.

2. The device of claim 1, wherein the force is configured to hold the articulator against the adjacent surface.

3. The device of claim 1, wherein the adjacent surface comprises skin and the movement is caused by a blood vessel residing beneath the skin.

4. The device of claim 1, wherein the articulator is configured to amplify the motion by translating the motion into a plurality of orientation changes in a plurality of planes.

5. The device of claim 1, wherein the circuitry is coupled to the accelerometer using a wire configured to carry an electrical signal.

6. The device of claim 1, further comprising a processor configured to distinguish between a plurality of types of motion data.

7. The device of claim 1, wherein the accelerometer is coupled to a post configured to extend outward from an edge of the articulator in a direction away from the pivot point.

8. The device of claim 1, wherein the articulator comprises a flat surface and a rounded surface, the rounded surface configured to be placed against the adjacent surface.

9. The device of claim 1, wherein the articulator is configured to be placed on a wrist such that the force is configured to occlude a blood vessel against a bone tissue.

10. The device of claim 9, wherein the articulator is configured to rotate about the pivot point in response to a radial force caused by a pulse running through a blood vessel.

11. The device of claim 1, further comprising another motion sensor configured to be placed in a second location on the adjacent surface different from a first location of the accelerometer, the another motion sensor configured to detect motion unrelated to the structure.

12-19. (canceled)
